(19) United States
(12) Patent Application Publication
(10) Pub. No.: US 2021/0298675 A1
(43) Pub. Date: Sep. 30, 2021

(54) IN-EAR CUSTOM BIOMETRIC HEARABLE AND METHOD OF MANUFACTURE

(71) Applicant: CUSTOM BIOMETRIC WEARABLES, INC., ATLANTA, GA (US)

(72) Inventor: SAMUEL B. KELLETT, JR., ATLANTA, GA (US)

(73) Assignee: CUSTOM BIOMETRIC WEARABLES, INC., ATLANTA, GA (US)

(21) Appl. No.: 16/835,257

(22) Filed: Mar. 30, 2020

(51) Int. Cl. A61B 5/00; A61B 5/0478; A61B 5/0205

(52) U.S. Cl. CPC A61B 5/6817; A61B 5/0478; A61B 5/1075; A61B 5/7475; A61B 5/0205

(57) ABSTRACT

Embodiments of the present invention include a custom biometric hearable including two in-ear custom biometric monitors including a custom biometric monitor shaped in dependence upon a user's right ear and a custom biometric monitor shaped in dependence upon the user's left ear; a data storage and charging unit; and a battery unit.

### BACKGROUND

Electroencephalography was used in a rudimentary form in dogs in 1912 by Ukrainian physiologist Vladimir Vladimirovich Pravdich-Neminsky, and then used by German Physiologist Hans Berger's first human EEG in 1924. Over the following century, its use has become mainstream and is now integral to the practice of neurology and other practices. Given the sampling bias inherent to routine EEG (typically 20-40 minutes in duration), the use of long-term continuous EEG (cEEG) has become exponentially more widespread, and it is now offered at most tertiary care medical centers. The use of inpatient cEEG occurs in both Intensive Care Units (ICUs) and Epilepsy Monitoring Units (EMUs) and is often used, for example, for the detection of seizures and epileptiform discharges, to localize epileptogenic foci for surgical resection, to distinguish seizures from non-epileptic events (e.g. psychogenic spells, syncope, migraine variants, movement disorders), to monitor depth of sedation, to prognosticate in certain situations, as an adjunct for brain-death testing, and to clarify potential etiologies of encephalopathy, among others.

Despite advances over the past century, cEEG remains a relatively costly and labor-intensive enterprise. Conventional inpatient monitoring, for instance, has a list price averaging of $3,856 per day, and with typical lengths of stay of 6 days, the cost of admissions may run in the tens of thousands of dollars. Furthermore, most clinics providing such services are limited to few beds and few admissions.

Ambulatory EEG (aEEG), or longer-term EEG offered in the outpatient setting, is a relatively recent advancement that has helped decrease the burden on inpatient cEEG as well as ease the bottleneck of admissions into the EMU. Conventional methods use wet electrodes on the scalp. After a relatively short period of time the wet electrodes—adhered to the scalp with a conductive gel or paste—begin to detach and the recording quality rapidly degrades.

Unfortunately, as important as it is, current outpatient EEG monitoring is limited. Conventional longer-term monitoring requires inpatient hospitalization, where EEG technologists can monitor the EEG tracings and repair electrodes when needed in this setting. There is an ongoing need for improvement in EEG monitoring.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 sets forth a line drawing of a custom biometric hearable according to example embodiments of the present invention.

FIG. 2 sets forth a line drawing of an in-ear biometric monitor useful with custom biometric hearables according to embodiments of the present invention. FIG. 2 also depicts that the anterior and posterior electrodes are used for measuring biometrics and the concha electrode is used for referencing.

FIG. 3 sets forth a line drawing of an example data storage and charging unit for use in example custom biometric hearables according to embodiments of the present invention.

FIG. 4 sets forth a line drawing of a battery unit useful with custom biometric hearables according to embodiments of the present invention.

FIG. 5 sets forth a line drawing of an in-ear biometric monitor oriented in a manner to illustrate the location of the anterior canal electrode.

FIG. 6 sets forth a line drawing of an in-ear biometric monitor oriented in a manner to illustrate the location of digital waxing in the anterior canal according to some embodiments of the present invention.

FIG. 7 sets forth a line drawing of an in-ear biometric monitor oriented in a manner to illustrate the location of the first bend corresponding to the first bend of the user's ear canal.

FIG. 8 sets forth a line drawing of an in-ear biometric monitor (102a and 102b) oriented in a manner to illustrate the location of the second bend (802) corresponding to the second bend of the user's ear canal.

FIG. 9 sets forth a line drawing of an in-ear biometric monitor oriented in a manner to illustrate the location of the posterior canal electrode corresponding to the anterior bend of the user's ear canal.

FIG. 10 sets forth a flow chart illustrating a method of manufacture of a custom in-ear monitor.

FIG. 11 sets forth a system diagram of an efficacy platform (1100) according to embodiments of the present invention.

### DETAILED DESCRIPTION

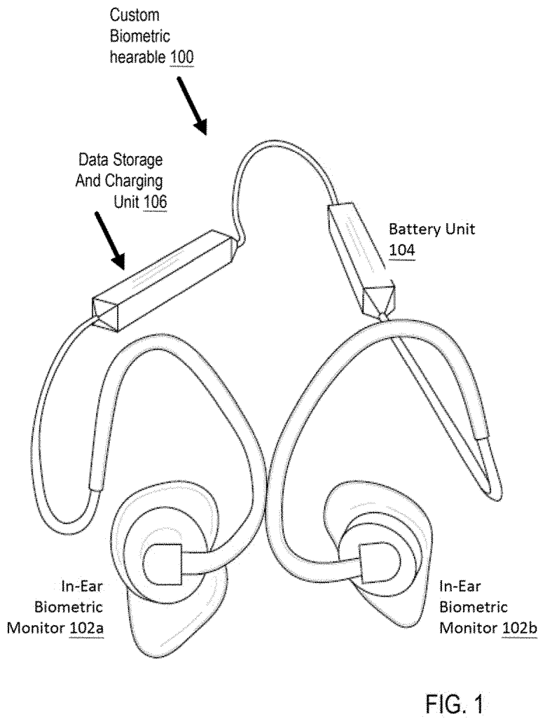

Systems, platforms, products, methods of manufacture and other features of and in support of custom biometric hearables according to embodiments of the present invention are described with reference to the accompanying drawings beginning with FIG. 1. FIG. 1 sets forth a line drawing of a custom biometric hearable according to example embodiments of the present invention. The custom biometric hearable (100) of FIG. 1 includes two in-ear custom biometric monitors (102a and 102b). In the example of FIG. 1, the in-ear custom biometric monitors are shaped in dependence upon the user's ear. That is, the user's ear is used in shaping the contours of the custom biometric monitor. That said, as will occur to those of skill in the art, although the custom biometric monitor is shaped in dependence upon the user's ear and is as such custom the term custom is not to suggest that the monitor must correspond perfectly with each contour of the user's ear. In fact, in some cases, it is preferable for performance of the custom biometric hearable to have a device that has slight deviances from the absolute shape of the user's ear.

In the example of FIG. 1, a custom biometric monitor (102a) is shaped in dependence upon a user's right ear and a custom biometric monitor (102b) is shaped in dependence upon the user's left ear. As will occur to those of skill in the art, the shape of the right ear and the left ear of a user may vary widely. As such, each in-ear biometric monitor is shaped in dependence upon the ear in which it is intended to reside while the custom biometric hearable (100) is in use.

The example custom biometric hearable (100) of FIG. 1 also includes a data storage and charging unit (106). The data storage and charging unit in the example of FIG. 1 provides an onboard unit for storage of data collected through the in-ear biometric monitors as well as providing ports for the transmission of that data and also for charging rechargeable batteries in the battery unit if such rechargeable units are in use.

As just mentioned above, the custom biometric hearable (100) of FIG. 1 also includes a battery unit (104). Such a battery unit may be designed to facilitate a rechargeable battery, a replaceable battery (rechargeable or otherwise), or a simple disposable battery as will occur to those of skill in the art.

The custom biometric hearable (100) of FIG. 1 advantageously provides anatomic proximity to the temporal lobes of the user.

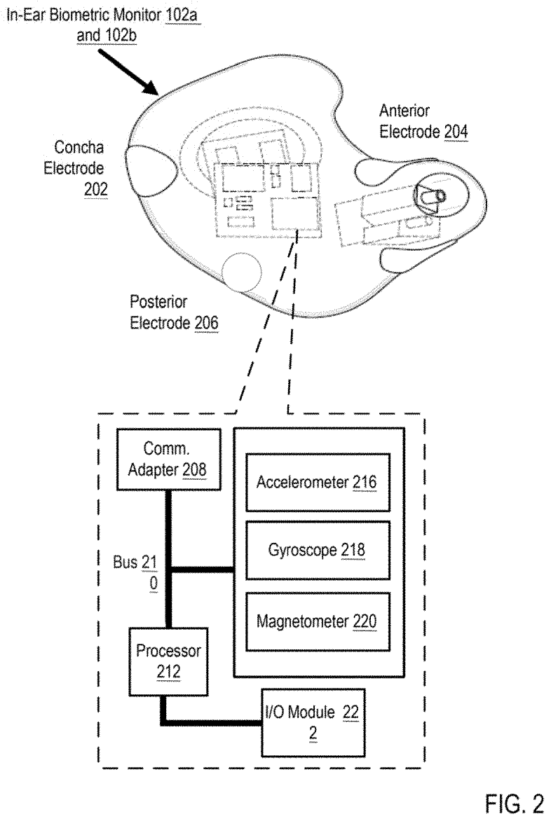

For further explanation, FIG. 2 sets forth a line drawing of an in-ear biometric monitor (102a and 102b) useful with custom biometric hearables according to embodiments of the present invention. The in-ear custom biometric monitors (102a and 102b) includes electrodes placed on the monitor at a concha position (202), an anterior position (204), and a posterior position (206) relative to the user's ear.

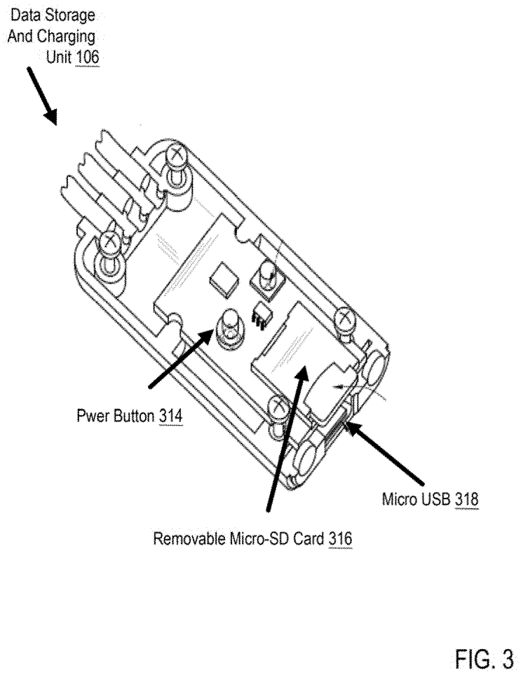

The example electrodes of FIG. 2 are implemented as dry electrodes. Dry electrodes are typically implemented as with metal that acts as a conductor between the skin and the electrode. These electrodes are dry in the sense that they do not require the use of an electrolytic gel material to facilitate that conduction. The example in-ear custom biometric monitors of FIG. 2 include an analog to digital converter chip (‘ADC converter chip’) (214) connected to a communications adapter (208), a processor (212), and I/O module, through a system bus (210). The example custom biometric monitors of FIG. 2 includes an accelerometer, a gyroscope, and a magnetometer. The accelerometer, gyroscope, and magnetometer of the ADC converter chip in the example of FIG. 2 are presented for explanation and not for limitation. In-ear custom biometric monitors according to embodiments of the present invention may include different, more, or less components as will occur to those of skill in the art. For example, in some embodiments, the custom biometric monitor contains an also a pulse oximeter and a fiber optic infrared radiometer to name only a few. For further explanation, FIG. 3 sets forth a line drawing of an example data storage and charging unit (106) for use in example custom biometric hearables according to embodiments of the present invention. In the example of FIG. 3, the data storage and charging unit (106) includes a power button (314), a removable Micro-SD card (316), and a Micro USB port. The power button, removable Micro-SD card, and Micro USB port are presented in this specification for explanation and not for limitation. In fact, similar functions may be implemented through different interfaces and technologies and all such interfaces and technologies are well within the scope of the present invention.

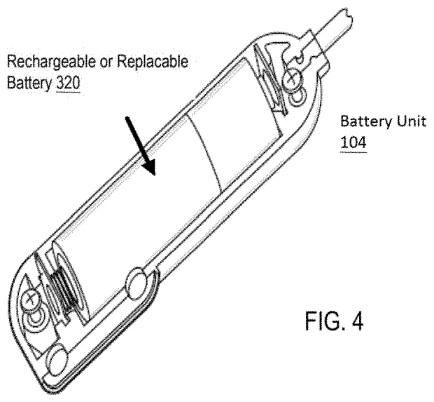

For further explanation, FIG. 4 sets forth a line drawing of a battery unit (104) useful with custom biometric hearables according to embodiments of the present invention. The example battery unit (104) provides an infrastructure (320) that is configured to accommodate a rechargeable battery, a replaceable battery (rechargeable or otherwise) or a disposable battery. While the example of FIG. 4 depicts a battery unit that is configured to accommodate multiple types of batteries, this is for explanation and not for limitation. In fact, battery units according to embodiments of the present invention may be configured to accommodate only a single type of battery or some other type of battery as will occur to those of skill in the art.

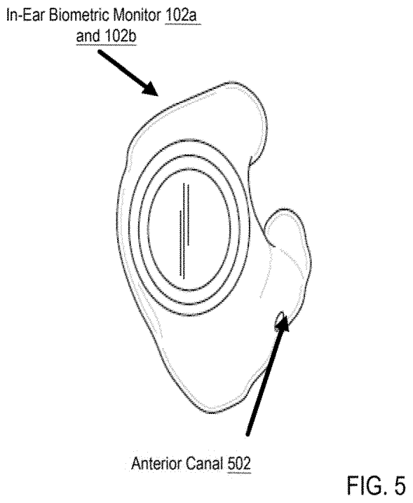

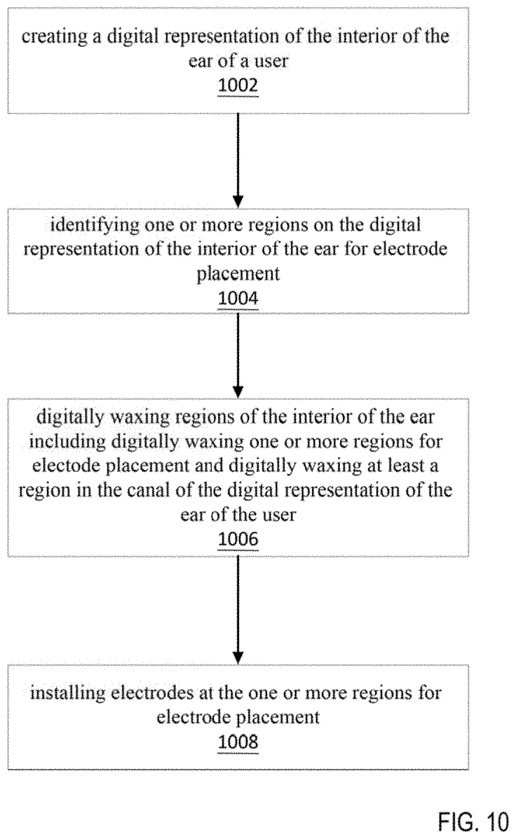

For further explanation, FIG. 5 sets forth a line drawing of an in-ear biometric monitor (102a and 102b) oriented in a manner to illustrate the location of the anterior canal (502). In some embodiments of the present invention, the anterior canal may be the location of an electrode. Furthermore, in some embodiments of the present invention, the location of the anterior canal may be a location for digital waxing. Such digital waxing may be carried out by increasing the volume of the monitor in such a location as discussed in more detail below with reference to FIG. 10.

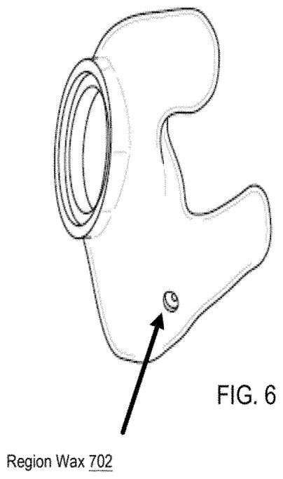

For further explanation, FIG. 6 sets forth a line drawing of an in-ear biometric monitor (102a and 102b) oriented in a manner to illustrate the location of the location of digital waxing in the anterior canal (502) according to some embodiments of the present invention. In some embodiments of the present invention, the anterior canal may be the location of an electrode and may be a location for digital waxing. Such digital waxing may be carried out by increasing the volume of the monitor in such a location as discussed in more detail below with reference to FIG. 10.

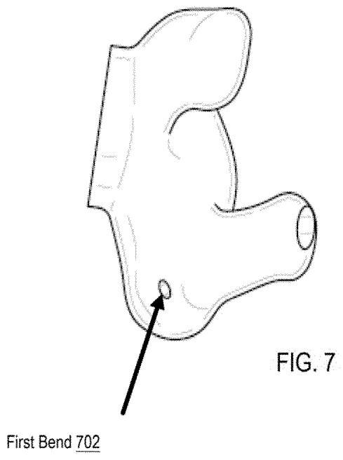

For further explanation, FIG. 7 sets forth a line drawing of an in-ear biometric monitor (102a and 102b) oriented in a manner to illustrate the location of the first bend (702) corresponding to the first bend of the user's ear canal. In some embodiments of the present invention, the first bend may be an indicator for the location of an electrode and may also be an indicator for a location for digital waxing. Such digital waxing may be carried out by increasing the volume of the monitor in such a location as discussed in more detail below with reference to FIG. 10.

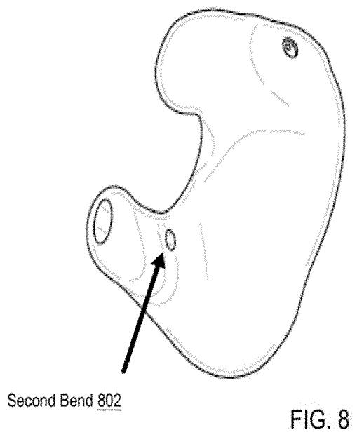

For further explanation, FIG. 8 sets forth a line drawing of an in-ear biometric monitor (102a and 102b) oriented in a manner to illustrate the location of the second bend (802) corresponding to the second bend of the user's ear canal. In some embodiments of the present invention, the second bend may be an indicator for the location of an electrode and may also be an indicator for a location for digital waxing. Such digital waxing may be carried out by increasing the volume of the monitor in such a location as discussed in more detail below with reference to FIG. 10.

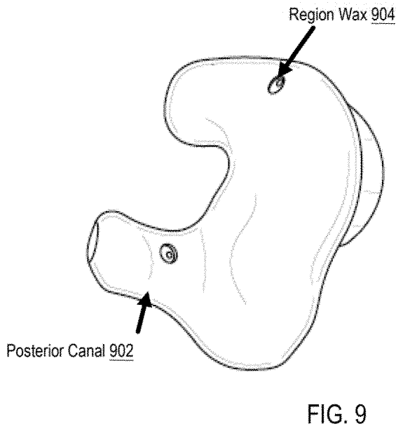

For further explanation, FIG. 9 sets forth a line drawing of an in-ear biometric monitor (102a and 102b) oriented in a manner to illustrate the location of the location of the posterior canal (902) corresponding to the posterior bend of the user's ear canal. In some embodiments of the present invention, the posterior canal may be the location of an electrode. Furthermore, in some embodiments of the present invention, the location of the posterior canal may be a location for digital waxing. Such digital waxing may be carried out by increasing the volume of the monitor in such a location as discussed in more detail below with reference to FIG. 10.

For further explanation, FIG. 10 sets forth a flow chart illustrating a method of manufacture of a custom in-ear monitor. Such an in-ear biometric monitor is useful with and will facilitate custom biometric hearables according to embodiments of the present invention.

The method of FIG. 10 includes creating (1002) a digital representation of the interior of the ear of a user. Creating a digital representation of the interior of the ear of a user includes scanning the interior of the ear with a non-invasive scanner. In some embodiments, scanning the interior of the ear with a non-invasive scanner according to methods and systems described in U.S. Pat. Nos. 8,900,126; 8,900,125; 8,900,129; 8,715,173; 8,900,128; 8,900,127; 8,900,130; 8,842,273; 9,188,775 and 8,841,603 all incorporated in their entirety herein by reference.

Creating a digital representation of the interior of the ear of a user according to the method of FIG. 10 may also include includes developing a physical mold of the interior of the ear and scanning the physical mold. Creating such a physical mold is often performed by an audiologist and the physical mold so created is then scanned to create the digital representation useful in embodiments of the present invention.

The method of FIG. 10 includes identifying (1004) one or more regions on the digital representation of the interior of the ear for electrode placement. In one embodiment of the present invention, identifying (1004) one or more regions on the digital representation of the interior of the ear for electrode placement includes identifying a concha region, an anterior region, and a posterior region. The identification of the concha region, an anterior region, and a posterior region are presented for explanation and not for limitation. In other embodiments, other regions may be identified for electrode placement as will occur to those of skill in the art.

The method of FIG. 10 includes digitally waxing (1006) regions of the interior of the ear including digitally waxing one or more regions for electrode placement and digitally waxing at least a region in the canal of the digital representation of the ear of the user. In some embodiments, digitally waxing regions of the interior of the ear includes identifying regions of the digital representation and increasing the volume of one or more of the identified regions. In some embodiments, digitally waxing regions of the interior of the ear includes identifying regions of the digital representation and increasing the volume of one or more of the identified regions includes identifying one or more locations on the digital representation for the placement of electrodes and increasing the volume of the digital representation at the one or more locations.

In some embodiments of the method of FIG. 10 increasing the volume of the digital representation includes adding an additional 0.25 mm of increased thickness to the area between the canal opening and the second bend of the canal. In some embodiments of the method of FIG. 10, increasing the volume of the digital representation includes adding an additional thickness of 0.3 mm to each electrode region such as for example in the anterior canal at or before second bend; posterior canal at or before second bend; and the upper posterior undercut of the concha bowl.

The method of FIG. 10 includes installing (1008) electrodes at the one or more regions for electrode placement. In some embodiments of the present invention, installing (1008) electrodes at the one or more regions for electrode placement includes installing three electrodes including installing an anterior canal electrode in a location at and before second bend, installing a posterior canal electrode at a location before the second bend; and installing a concha electrode in the upper posterior undercut electrode of concha bowl. The concha electrode is used in some embodiments as a reference electrode as will occur to those of skill in the art.

Installing electrodes at the one or more regions for electrode placement may be carried out by determining distance between the one or more electrodes and installing electrodes according to a predetermined distance constraint. In some example embodiments of the method of FIG. 10, the distance between the one or more electrodes may follow the following constraints: at least 2 mm between all installed electrodes including post-installation adaption of electrode and 10 mm in diameter of each and any electrode along the length of the canal. The example constraints are provided for explanation and not for limitation. In fact, many other constraints may occur to those of skill in the art and all are well within the scope of the present invention.

Installing electrodes at the one or more regions for electrode placement may be carried out by electroplating at a portion of the one or more regions for electrode placement. Electroplating is a process that uses an electric current to reduce dissolved metal cations so that they form a thin coherent metal coating on an electrode.

Installing electrodes according to a predetermined distance constraint may include depositing electrode material at the identified region; and removing portions of the electrode material according to the constraints. Removing portions of the electrodes material according to the constraints may be carried out by physically cutting away portions of the material, using machines to removed portions, and in other ways as will occur to those of skill in the art.

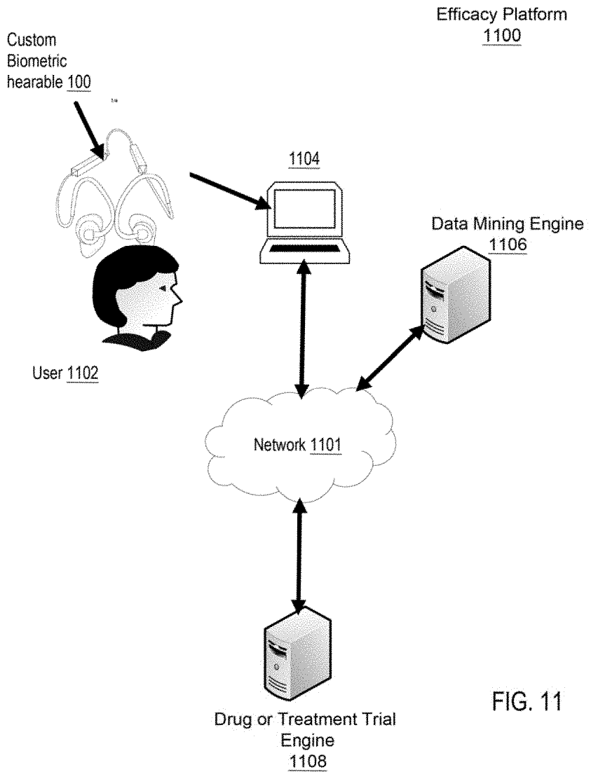

Custom biometric hearables according to embodiments of the present invention have wide-spread implications. For example, such custom biometric hearables may be used in drug or treatment efficacy programs. For further explanation, therefore, FIG. 11 sets forth a system diagram of an efficacy platform (1100) according to embodiments of the present invention.

In the example of FIG. 11, a user (1102) wears a custom biometric hearable (100) such as those constructed according to embodiments of the present invention which in turn transmits data from the user to a local computer (1104). Such transmissions may be wired or wireless, real-time, or in an interval fashion. That local computer may be accessed by a local practitioner who is conducting a local trial for drug or treatment efficacy and the like.

In the example of FIG. 11, such data is then transmitted to a drug or treatment trial engine (1108). The drug or treatment trial engine (1108) represents the reservoir of information obtained from many users, infrastructure to analyze such data and infrastructure to determine the efficacy of the drug or treatment.

In the example of FIG. 11, the efficacy platform (1100) also includes a data mining engine (1106). Such a data mining engine is coupled for data communications with the drug or treatment trial engine (1108) and provides additional data mining support for determining the efficacy of a drug or treatment.

It should be noted while the orientation of the current disclosure is directed toward EEG, this is for explanation and not for limitation. Embodiments of the present invention may be used in conjunction with EKG, EMG, deep brain monitoring, and many other uses as will occur to those of skill in the art. The custom biometric monitor inside the ear also contains an accelerometer and gyroscope, a pulse oximeter, and a fiber optic infrared radiometer. The EEG reading coupled with the accelerometer and gyroscope can provide real sleep staging through the Delta brain wave to determine the amount of REM and other “quality of sleep” metrics. The pulse oximeter can be used to measure pulse and blood oxygenation to determine if breathing difficulties are being experienced by the wearer. The fiber optic infrared radiometer can be used to measure body temperature off of the tympanic membrane. Knowing the quality of sleep, the body temperature, and how well a person is breathing could be used to help determine if someone should seek further medical attention.

FIGS. 1-11 illustrate the architecture, functionality, and operation of possible implementations of platforms, systems, methods and products according to various embodiments of the present invention. In this regard, each block in a flowchart or block diagram may represent a module, segment, or portion of code or other automated computing machinery, which comprises one or more executable instructions or logic blocks for implementing the specified logical function(s). It should also be noted that, in some alternative implementations, the functions noted in the block may occur out of the order noted in the figures. For example, two blocks shown before or after one another may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method of manufacture of a custom in-ear monitor, the method comprising:
creating a digital representation of the interior of the ear of a user;
identifying one or more regions on the digital representation of the interior of the ear for electrode placement;
digitally waxing regions of the interior of the ear including digitally waxing one or more regions for electrode placement and digitally waxing at least a region in the canal of the digital representation of the ear of the user; and
installing electrodes at the one or more regions for electrode placement.

2. The method of claim 1 wherein installing electrodes at the one or more regions for electrode placement includes electroplating at least a portion of the one or more regions for electrode placement.

3. The method of claim 1 wherein installing electrodes at the one or more regions for electrode placement further comprises determining distance between the one or more electrodes and installing electrodes according to a predetermined distance constraint.

4. The method of claim 3 wherein installing electrodes according to a predetermined distance constraint further comprises:
depositing electrode material at the identified region; and
removing portions of the electrode material according to the constraints.

5. The method of claim 1 wherein creating a digital representation of the interior of the ear of a user includes scanning the interior of the ear with a non-invasive scanner.

6. The method of claim 1 wherein creating a digital representation of the interior of the ear of a user includes developing a physical mold of the interior of the ear and scanning the physical mold.

7. The method of claim 1 wherein digitally waxing regions of the interior of the ear includes identifying regions of the digital representation and increasing the volume of one or more of the identified regions.

8. The method of claim 1 wherein digitally waxing regions of the interior of the ear includes identifying regions of the digital representation and increasing the volume of one or more of the identified regions further comprises:
identifying one or more locations on the digital representation for the placement of electrodes; and
increasing the volume of the digital representation at the one or more locations.

9. A custom biometric hearable comprising:
two in-ear custom biometric monitors including a custom biometric monitor shaped in dependence upon a user's right ear and a custom biometric monitor shaped in dependence upon the user's left ear;
a data storage and charging unit; and
a battery unit.

10. The custom biometric hearable of claim 9 wherein each of the two in-ear custom biometric monitors includes electrodes placed on the monitor at a concha position, an anterior position, and a posterior position and the two anterior and two posterior electrodes are used for measuring while the two concha electrodes are used for referencing on the opposite custom biometric monitor.

11. The custom biometric hearable of claim 9 wherein at least one of the two in-ear custom biometric monitors includes an accelerometer.

12. The custom biometric hearable of claim 9 wherein at least one of the two in-ear custom biometric monitors includes a gyroscope.

13. The custom biometric hearable of claim 9 wherein at least one of the two in-ear custom biometric monitors includes a magnetometer.

14. The custom biometric hearable of claim 9 contains a pulse oximeter.

15. The custom biometric hearable of claim 9 contains a fiber optic infrared radiometer

16. The custom biometric hearable of claim 9 wherein the data storage and charging unit includes a power button.

17. The custom biometric hearable of claim 9 wherein the data storage and charging unit includes a removable Micro-SD card.

18. The custom biometric hearable of claim 9 wherein the data storage and charging unit includes a Micro-USB port.

19. The custom biometric hearable of claim 9 wherein the battery unit is configured to accommodate a rechargeable battery.

20. The custom biometric hearable of claim 9 wherein the battery unit is configured to accommodate a replaceable battery.

21. The custom biometric hearable of claim 18 wherein the replaceable battery is a disposable battery.

22. An efficacy platform comprising,
a custom biometric hearable; and
a trial engine.
